(19) United States
(12) Patent Application Publication
(10) Pub. No.: US 2024/0156930 A1
(43) Pub. Date: May 16, 2024

(54) TUMOR VACCINE BASED ON TUMOR-DERIVED MICROPARTICLES, AND PREPARATION METHOD AND USE THEREOF

(71) Applicant: UNION HOSPITAL TONGJI MEDICAL COLLEGE HUST, Wuhan (CN)

(72) Inventors: Yang JIN, Wuhan (CN); Qi TAN, Wuhan (CN); Zimo YANG, Wuhan (CN); Jiangbin CHEN, Wuhan (CN); Wenjuan CHEN, Wuhan (CN)

(21) Appl. No.: 18/477,664

(22) Filed: Sep. 29, 2023

(30) Foreign Application Priority Data
Nov. 14, 2022 (CN) 202211426392.9

(51) Int. Cl. A61K 39/00; A61K 9/16

(52) U.S. Cl. CPC A61K 39/0011; A61K 9/1641; A61K 9/1652; A61K 9/1682; A61K 2039/5152

(57) ABSTRACT

Disclosed is a tumor vaccine based on tumor-derived microparticles (TMPs), and a preparation method and use thereof. The preparation method includes the following steps: 1, subjecting tumor cells to separation to obtain TMPs; 2, mixing the TMPs with 0.03 μg/μl of a polyethyleneimine solution to allow a reaction for 10 min to 20 min, and washing to obtain PEI-modified TMPs (TMPs-PEI); and 3, mixing the TMP-PEI with 0.1 μg/μl of a lipopolysaccharide solution to allow cross-linking for 15 min to 1 h to obtain the tumor vaccine based on TMPs (TMP-PEI-LPS). A nanomaterial prepared by conducting surface modification on the TMPs with an immunologic adjuvant can be disguised as a bacterial analog to promote the recognition of tumor antigens in vivo. Moreover, the TMPs can retain their immunostimulatory ability and reduce their toxicity by using a cross-linking agent coupled with the immunologic adjuvant.

### CROSS-REFERENCE TO RELATED APPLICATION

This patent application claims the benefit and priority of Chinese Patent Application No. 202211426392.9 filed with the China National Intellectual Property Administration on Nov. 14, 2022, the disclosure of which is incorporated by reference herein in its entirety as part of the present application.

### TECHNICAL FIELD

The present disclosure relates to the technical field of tumor therapeutic drugs, in particular to a tumor vaccine based on tumor-derived microparticles (TMPs), and a preparation method and use thereof.

### BACKGROUND

Tumor vaccines are mainly composed of tumor antigens, appropriate adjuvants, and effective delivery vehicles, and supplemented by effective delivery methods. According to the different delivery forms of antigens, tumor vaccines can be divided into three categories at present, including nucleic acids that can encode antigens, proteins of tumor antigens, and engineered cells that can load or express cancer antigens. Tumor vaccines can promote the recognition of tumor antigens through different approaches, thereby enhancing anti-tumor immunity to inhibit tumor growth or even eliminate tumors. At present, the design of tumor vaccine mainly depends on the following methods. Nucleic acid containing tumor-associated antigens is designed and transfected into cells by electroporation and other transfection methods to express tumor antigens and then enhance tumor immunity. Peptide- or protein-based vaccines are screened or computer-designed to form proteins or polypeptides that are related to tumor immunity or can affect the biological characteristics of tumors, so as to enter the tumor and play a role to kill the tumor. Engineered cell-based vaccines operate on autologous or allogeneic immune cells in vitro (such as stimulation by co-incubation with antigens or antigen loading), and then infuse these cells back into the body for tumor treatment.

The limitations of current tumor vaccines mainly include the following factors:

(1) Low immunogenicity of the vaccine: the low expression of tumor antigens or the disappearance of antigenic epitopes leads to the low immunogenicity of the vaccine. (2) The vaccine delivery system affects the effect of tumor vaccines: tumor vaccines need effective delivery vectors to be presented to immune cells to function. (3) The tumor vaccines have high research and development costs and long synthesis time.

### SUMMARY

In view of the above problems and requirements, a technical problem to be solved by the present disclosure is to provide a tumor vaccine based on tumor-derived microparticles (TMPs), and a preparation method and use thereof.

To solve the above technical problem, the following technical solutions are adopted in the present disclosure.

The present disclosure provides a preparation method of a tumor vaccine based on TMPs, including the following steps:
step 1, subjecting tumor cells to separation to obtain TMPs;
step 2, mixing the TMPs with 0.02 μg/μl to 0.04 μg/μl of a polyethyleneimine (PEI) solution to allow a reaction for 10 min to 20 min, and washing the microparticles to obtain PEI-modified TMPs (TMPs-PEI); and
step 3, mixing the TMP-PEI with 0.05 μg/μl to 0.15 μg/μl of a lipopolysaccharide (LPS) solution to allow cross-linking for 15 min to 1 h to obtain the tumor vaccine based on TMPs (TMP-PEI-LPS).

Further, the TMPs are obtained by subjecting the tumor cells to ultraviolet irradiation and then the separation by gradient centrifugation in step 1.

Further, the reaction is conducted at 4° C. for 15 min, and the PEI solution has a concentration of 0.03 μg/μl in step 2.

Further, the reaction is conducted at 4° C. for 30 min, and the LPS solution has a concentration of 0.1 μg/μl in step 3.

Further, the present disclosure provides a tumor vaccine based on TMPs, which is prepared by the preparation method.

Further, the present disclosure provides use of the tumor vaccine based on TMPs in preparation of a drug for treating a tumor.

Compared with the prior art, the present disclosure adopting the above technical solutions has the following advantages:
(1) In the present disclosure, the tumor vaccine is designed based on TMPs because the TMPs cover the antigenic information on tumor cells, including the information on nucleic acid and protein. Therefore, TMPs are individualized whole-cell antigens superior to whole-cell lysates and exosomes. Moreover, the TMPs have a small particle size, which is beneficial for antigen-presenting cells to take up tumor antigens.
(2) Tumor vaccines need effective delivery vectors to be presented to immune cells to function. In the present disclosure, TMPs are used as tumor antigen carriers. The TMPs are desirable delivery vehicles for tumor antigens, with excellent histocompatibility and immune organ targeting property. The TMPs not only act as tumor antigens, but also as a carrier to deliver tumor antigens, thus contributing to the uptake and presentation of tumor antigens. Meanwhile, the surface-modified cross-linking agent has a charge adsorption effect, which can promote the uptake of tumor antigens more efficiently.
(3) Modification of TMPs with a cross-linking agent not only promotes the coupling of the immune adjuvant to the tumor antigen, but also promotes the effective uptake of the tumor antigen by the antigen-presenting cells by using the charge adsorption of the cross-linking agent to exert an immunostimulatory effect. A nanomaterial prepared by conducting surface modification on the TMPs with an immunologic adjuvant can be disguised as a bacterial analog to promote the recognition of tumor antigens in vivo. Moreover, the TMPs can retain their immunostimulatory ability and reduce their toxicity by using a cross-linking agent coupled with the immunologic adjuvant.
(4) TMPs are both high-quality tumor antigens and desirable antigen delivery vehicles. Therefore, the synthesis of tumor vaccines based on TMPs only needs to modify the immune adjuvant. Accordingly, the research and development has low cost and short synthesis period, and is suitable for clinical application.

The present disclosure will be described in detail below in conjunction with the accompanying drawings and examples.

### BRIEF DESCRIPTION OF THE DRAWINGS

FIG. 1A shows a process of manufacturing TMPs by ultraviolet irradiation and gradient centrifugation and a transmission electron microscope (TEM) image of the TMPs; FIG. 1B shows expression results of CD63, TSG101, and Calnexin of the TMPs detected by Western Blot; and FIG. 1C is a schematic diagram of distribution of the TMPs detected by DLS.

FIG. 2A shows a schematic diagram of synthesis of TMP-PEI-LPS and FIG. 2B shows an image of successful coupling of FITC-labeled LPS and DiI-labeled TMPs through fluorescence identification; FIG. 2C is a schematic diagram of efficiency of the synthesis of the TMP-PEI-LPS with different concentrations of PEI detected by flow cytometric analysis; FIG. 2D is a protein expression profile of the TMP-PEI-LPS synthesized with different concentrations of PEI analyzed by Coomassie Brilliant Blue; FIG. 2E shows a polydispersity index (PDI) of the TMP-PEI-LPS synthesized with different concentrations of PEI detected by DLS analysis; FIG. 2F is a particle size diagram of the TMP-PEI-LPS synthesized with different concentrations of PEI detected by DLS analysis; and FIG. 2G is a schematic diagram of a Zeta potential of the TMP-PEI-LPS synthesized with different concentrations of PEI detected by DLS analysis.

FIG. 3A is a light microscope image of dendritic cells DC2.4 uptake of DiI-labeled TMP-PEI-LPS; FIG. 3B is a schematic diagram of detecting an efficiency of DC2.4 uptake of TMP-PEI-LPS by flow cytometric analysis; FIG. 3C and FIG. 3D are schematic diagrams of results of detecting TMP-PEI-LPS stimulation of DC2.4 CD80 and CD86 by flow cytometry; FIG. 3E and FIG. 3F are schematic diagrams of results of detecting CD80 and CD86 in mouse primary cells stimulated by TMP-PEI-LPS by flow cytometry; FIG. 3G is a schematic diagram of results of ELISA detection of TMP-PEI-LPS stimulated DC2.4 to secrete TNF-α; FIG. 3H is a schematic diagram of results of ELISA detection of TMP-PEI-LPS stimulated mouse primary cells to secrete TNF-α; FIG. 3I is a schematic diagram of results of flow cytometric analysis detecting that TMP-PEI-LPS can induce tumor cell apoptosis by stimulating the macrophage cell line RAW264.7; and FIG. 3J is a schematic diagram of results of ELISA detection of TMP-PEI-LPS stimulated macrophage cell line RAW264.7 to secrete TNF-α.

FIG. 4A is an in vivo imaging diagram of lymph node TMP-PEI-LPS; FIG. 4B is a schematic diagram of the hyperplasia of bilateral inguinal lymph nodes stimulated by TMP-PEI-LPS; FIG. 4C is a schematic diagram of detecting the uptake of TMP-PEI-LPS by bilateral inguinal lymph nodes through flow cytometry analysis; FIG. 4D shows results of immunofluorescence detection of CD86 expression in lymph nodes promoted by TMP-PEI-LPS; FIG. 4E is a schematic diagram of the uptake of TMP-PEI-LPS by splenocytes detected by flow cytometry, and the uptake by dendritic cells in lymph nodes; FIG. 4F is mouse liver and kidney function indicators (ALT, AST, BUN, and CREA) after TMP-PEI-LPS injection; and FIG. 4G is HE slice diagram of mouse heart, lung, liver, and kidney tissue after TMP-PEI-LPS injection; and

FIG. 5A is a schematic diagram of the mouse experiment process; FIG. 5B is a schematic diagram of a body weight change of the experimental mice during the intervention; FIG. 5C is a schematic diagram of an influence of TMPs-PE I-LPS on tumor growth; FIG. 5D is a schematic diagram of a change in tumor load volume of each mouse in different groups; and FIG. 5E is a schematic diagram of a change in the spleen index of mice in different groups.

### DETAILED DESCRIPTION OF THE EMBODIMENTS

The principles and features of the present disclosure are described below with reference to the accompanying drawings. The listed embodiments are only used to explain the present disclosure, rather than to limit the scope of the present disclosure.

### I. Preparation and Identification of Toll-Like Receptor 4 Agonist LPS-Modified TMPs (Tmp-Pei-Lps)

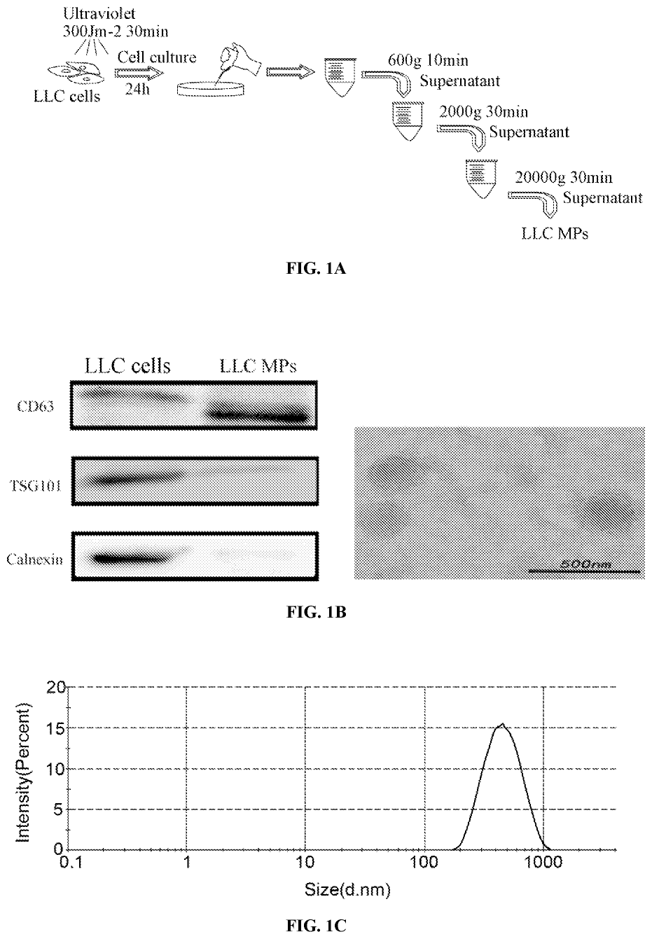

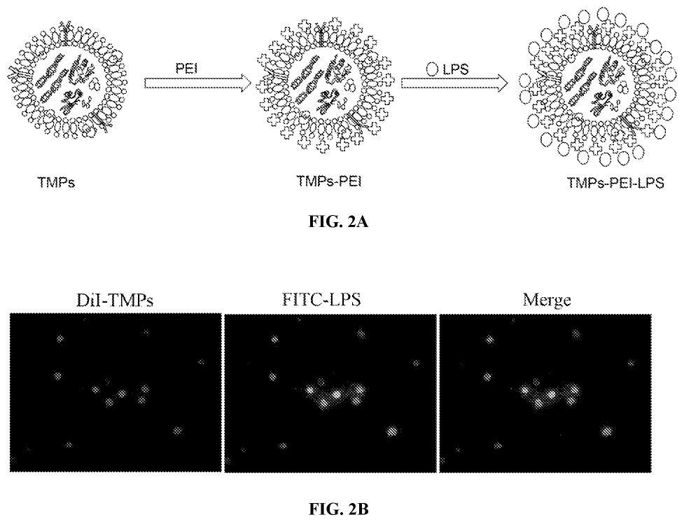

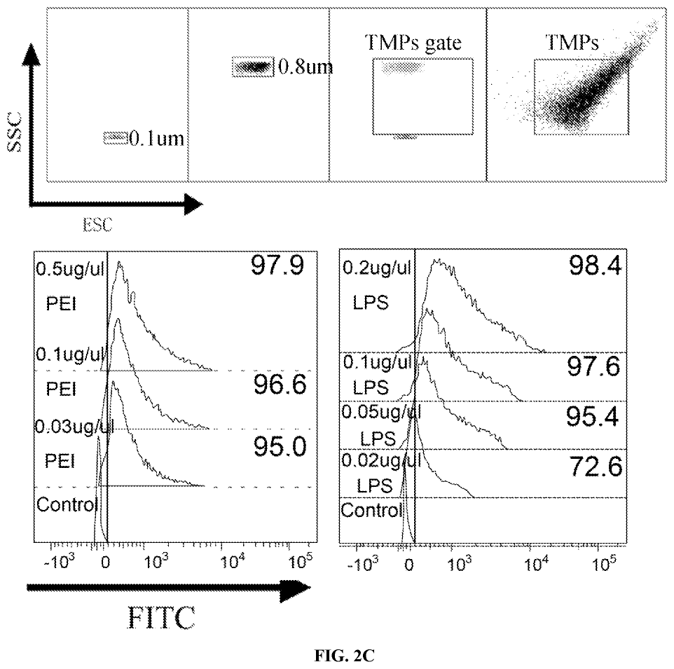

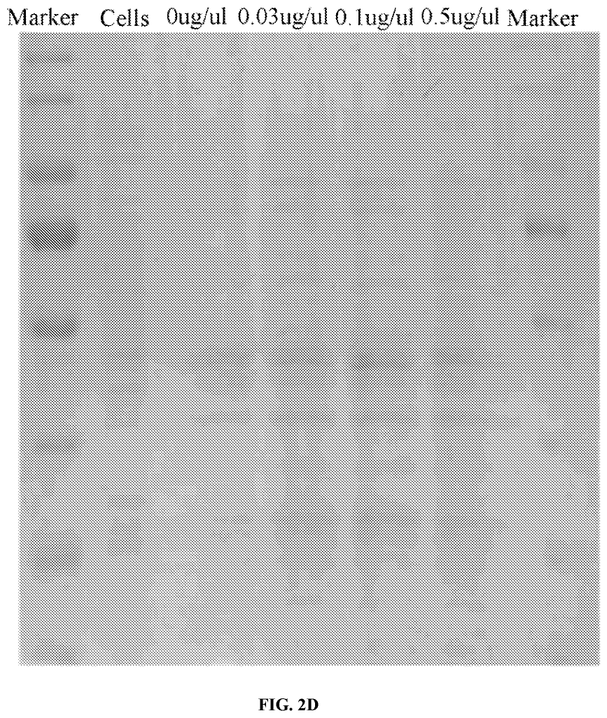

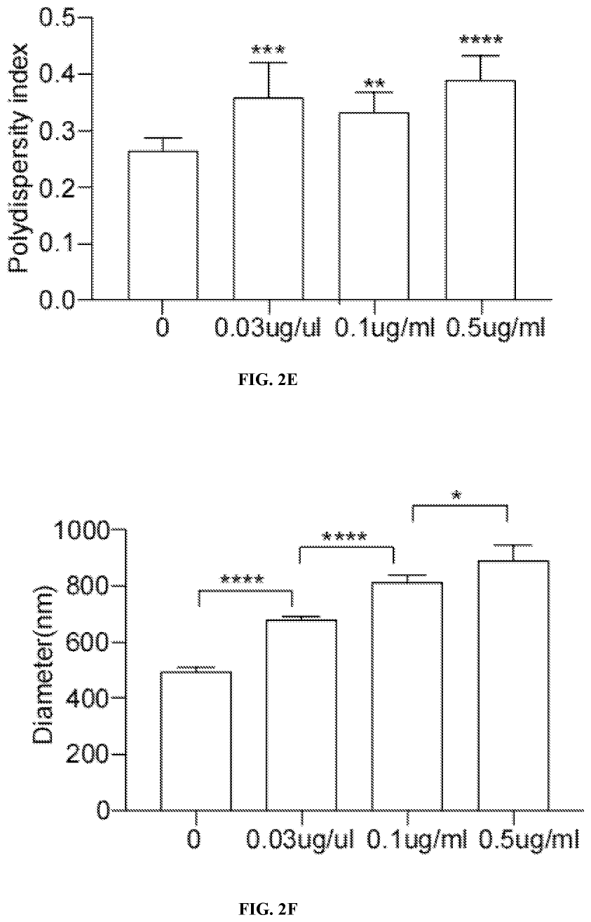

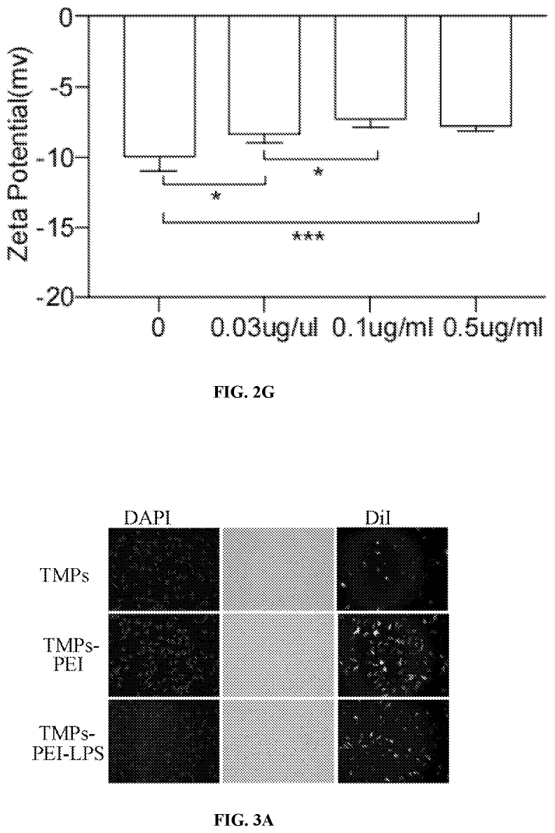

TMPs were isolated by ultraviolet irradiation and gradient centrifugation, and observed by TEM to confirm a membrane structure of the phospholipid bilayer (FIG. 1A). The detection of positive markers (CD63 and TSG101) and negative marker (Calnexin) of extracellular vesicles by Western-blot technique confirmed that TMPs had been successfully isolated (FIG. 1B). Particle size analysis confirmed that the extracted TMPs had particle sizes mainly distributed in a range of 100 nm to 1,000 nm, which met the particle size standard of TMPs. By utilizing the function of PEI as a cationic cross-linker, surface-negatively-charged TMPs were cross-linked with negatively-charged lipid A-enriched LPS via charge adsorption (FIG. 2A). TMPs with the same number of particles were cross-linked with different concentrations of PEI, and it was found that a binding efficiency between LPS and TMPs increased with an increase of PEI concentration (FIG. 2B). Coomassie brilliant blue protein assay was conducted to analyze the protein expression of different concentrations of synthetic vaccines. It was found that the concentration of PEI did not affect the expression of the vaccine protein (FIG. 2C). The particle size and zeta potential of synthetic vaccines with different concentrations of PEI were then compared. It was found that the surface modification of PEI significantly reduced the zeta potential of the vaccine (FIG. 2E). Analysis of particle size showed that the particle size of the vaccine synthesized with 0.1 μg/ml and 0.5 μg/ml of PEI was significantly increased (FIG. 2F). Combining the above results, TMP-PEI-LPS was synthesized with 5×10⁵ TMPs, 0.03 μg/μl PEI, and 0.1 μg/μl LPS.

### II. In Vitro Verification of TMP-PEI-LPS Stimulating Maturation of Dendritic Cells and Polarization of Macrophages M1

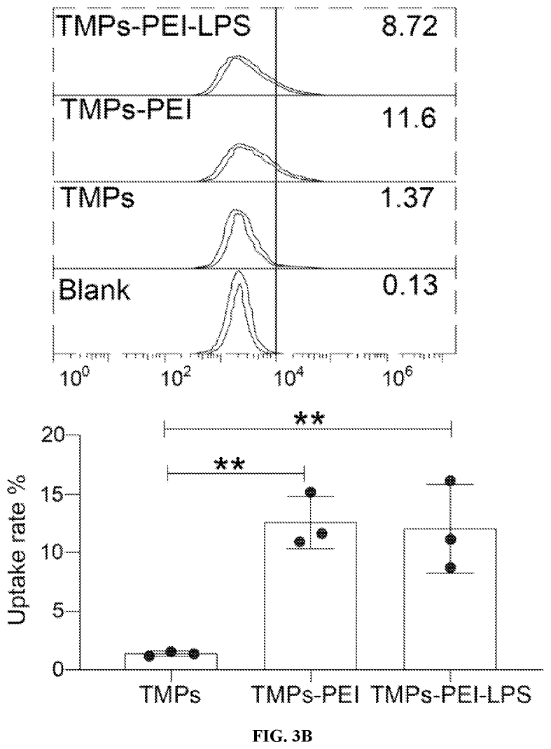

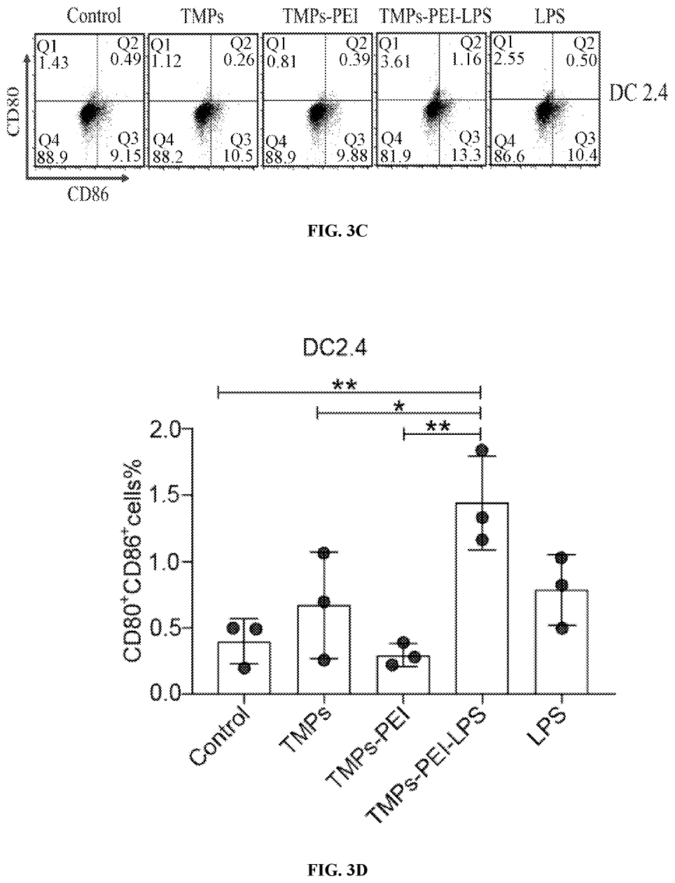

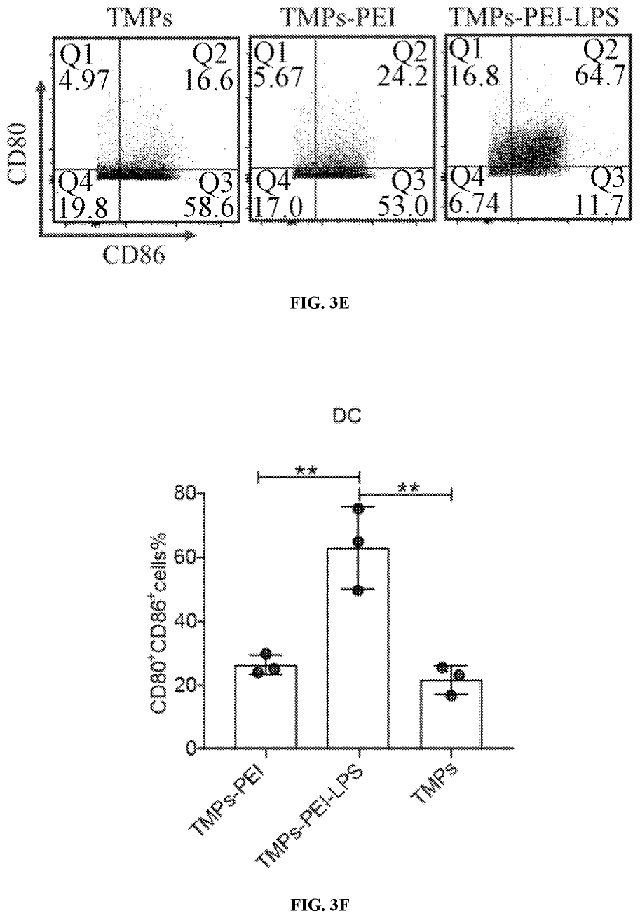

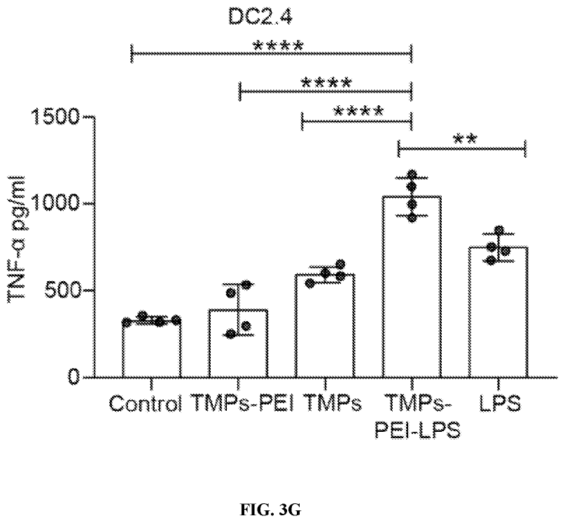

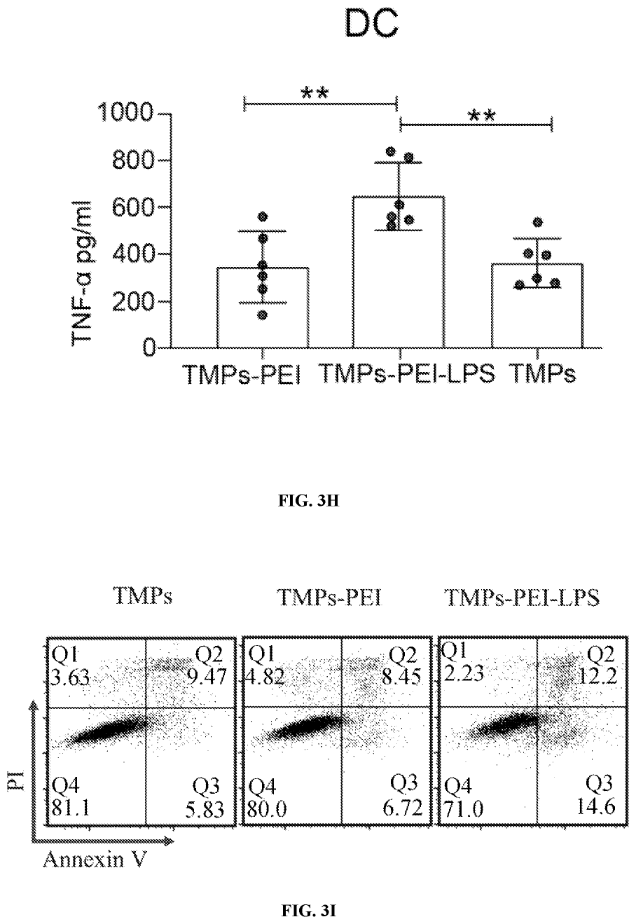

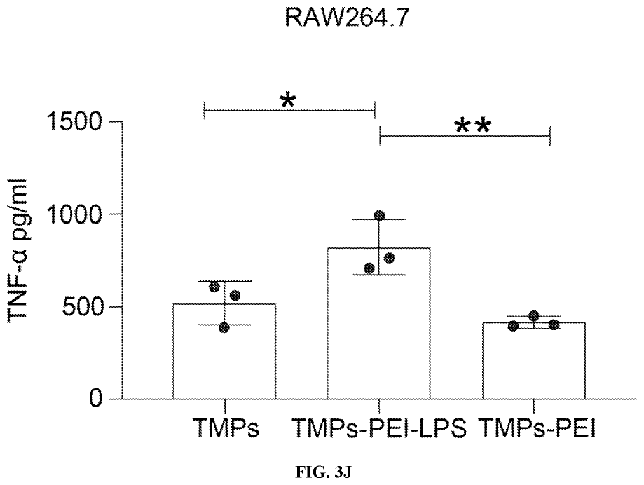

TMPs, TMP-PEI, and TMP-PEI-LPS were labeled with DiI, incubated with DC2.4 cells for 3 h, and then compared. The uptake capacity of the DC2.4 was assessed by fluorescence microscopy (FIG. 3A). The results showed that the uptake of cells in TMP-PEI group and TMP-PEI-LPS group was higher than that in TMPs group. Further experiments verified the immune activation ability of TMP-PEI-LPS dendritic cells. It was found that TMP-PEI-LPS significantly stimulated the upregulation of CD80 and CD86 in DC2.4 cell line and mouse bone marrow primary dendritic cells, and stimulated the increased secretion of TNF-α (FIG. 3C to FIG. 3G). Further, the immune stimulation ability of TMP-PEI-LPS was explored on macrophages. It was found that TMP-PEI-LPS could significantly stimulate the macrophage cell line RAW264.7 to secrete TNF-α, and significantly induce the apoptosis of lung adenocarcinoma cells (FIG. 3I to FIG. 3J). The results demonstrated that the TMP-PEI-LPS stimulated the maturation of dendritic cells and the polarization of M1 macrophages.

### III. Animal Experiment Verification of TMP-PEI-LPS Targeting DCs and Stimulating their Maturation

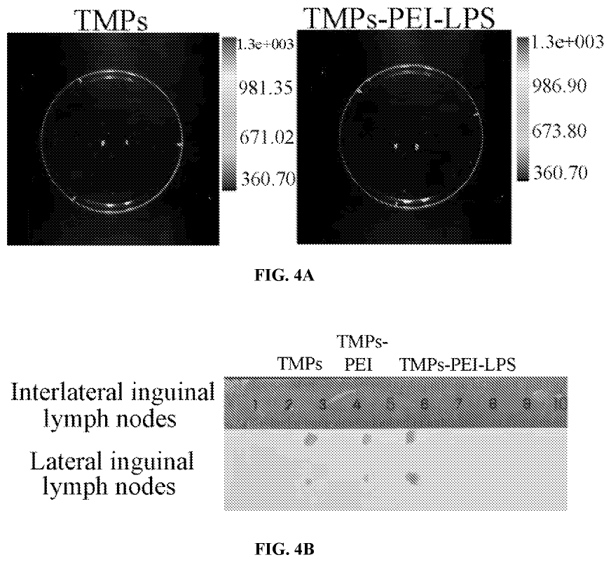

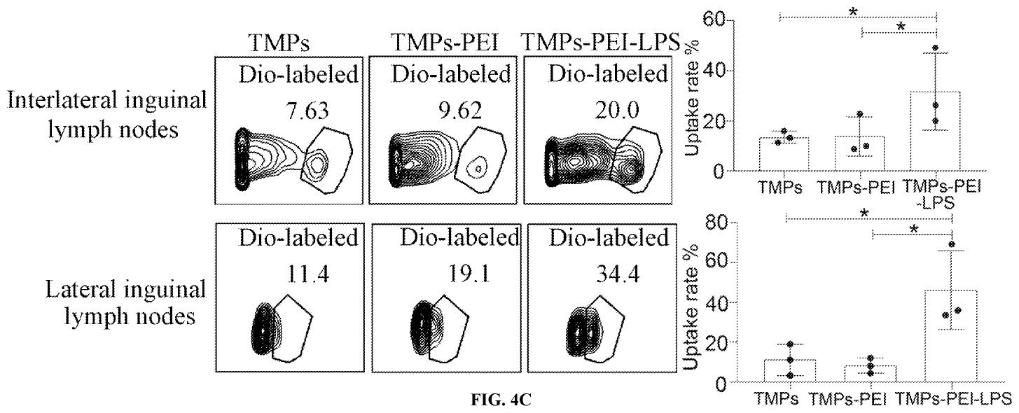

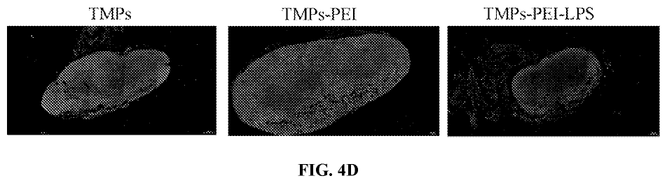

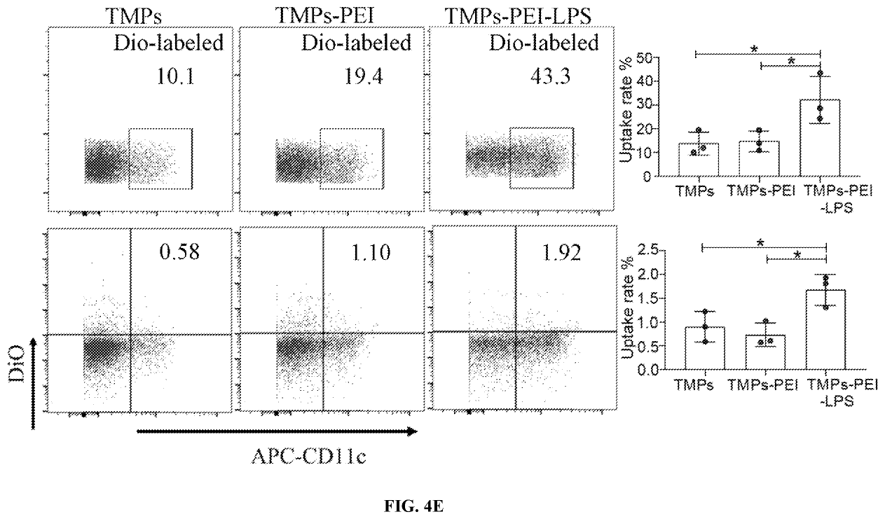

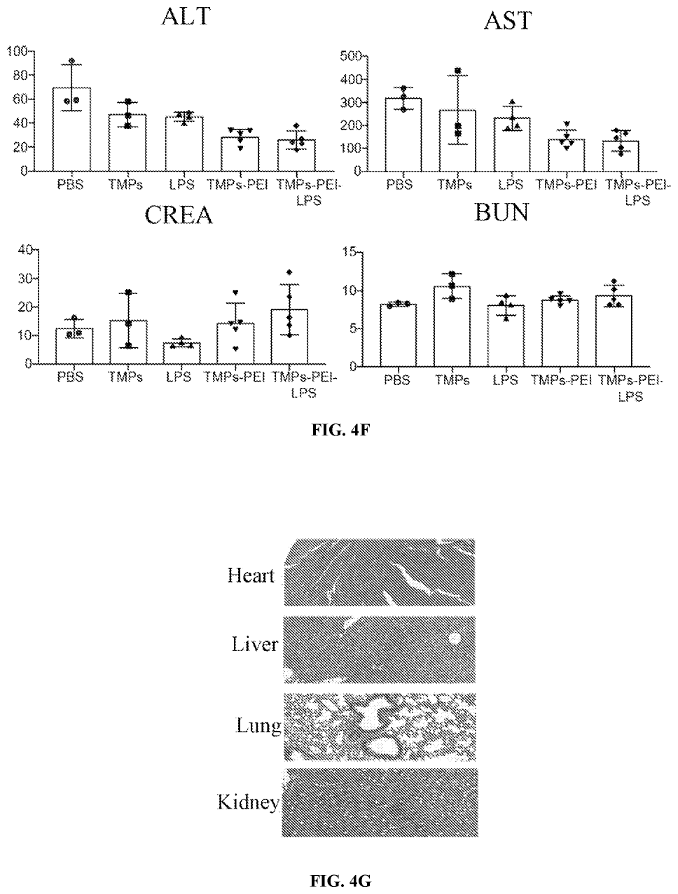

In order to test the absorption of the prepared nanomaterials in vivo, TMP-PEI-LPS and TMPs were separately injected into the left back and groin of mice. After 24 h of stimulation, bilateral lymph nodes were removed for in vivo imaging. The results showed that the TMP-PEI-LPS could be taken up by lymph nodes in a short time and drained to the contralateral lymph nodes (FIG. 4A). Likewise, the TMP-PEI-LPS could stimulate the enlargement of bilateral lymph nodes and promote the rapid uptake by dendritic cells and the drainage into the lymph nodes (FIG. 4B and FIG. 4C). Immunofluorescence detection of lymph nodes found that the TMP-PEI-LPS could stimulate the expression of CD86 in lymph nodes, suggesting that the TMP-PEI-LPS could target dendritic cells and stimulate their maturation, thus completing lymph node homing (FIG. 4D). The uptake of TMPs-PE I-LPS by spleen cells was found to be increased by tail vein injection, especially by dendritic cells (FIG. 4E). The safety of the prepared material was checked by testing the mice for liver and kidney function, and by analyzing slices of major organs, including heart, liver, spleen, lung, and kidney. The results showed that the injection of TMP-PEI-LPS did not cause significant damage to the liver and kidney. In addition, tissue sections showed no obvious pathological changes after injection. These results demonstrated the safety of the anti-tumor vaccine of the present disclosure in vivo (FIG. 4F to FIG. 4G). IV. In vitro verification of TMP-PEI-LPS promoting T cell activation and killing tumors

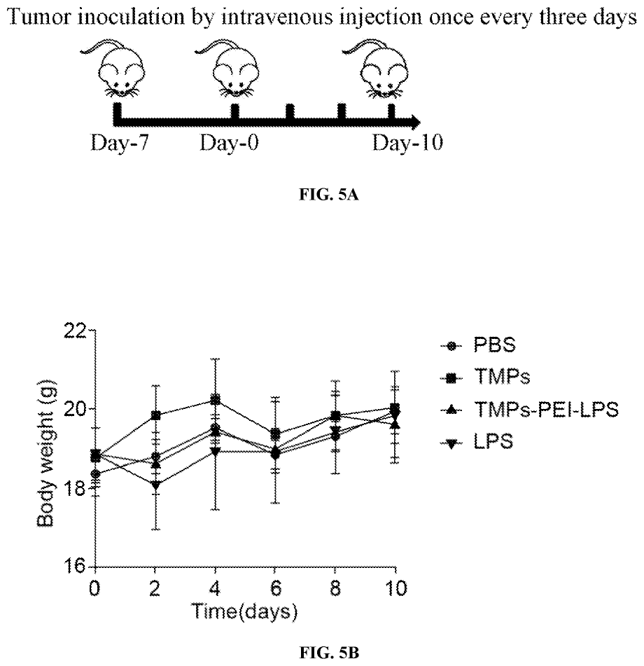

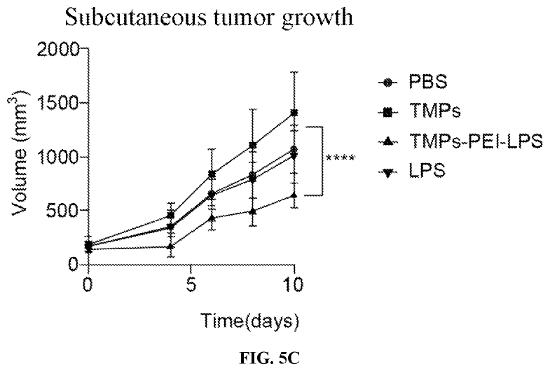

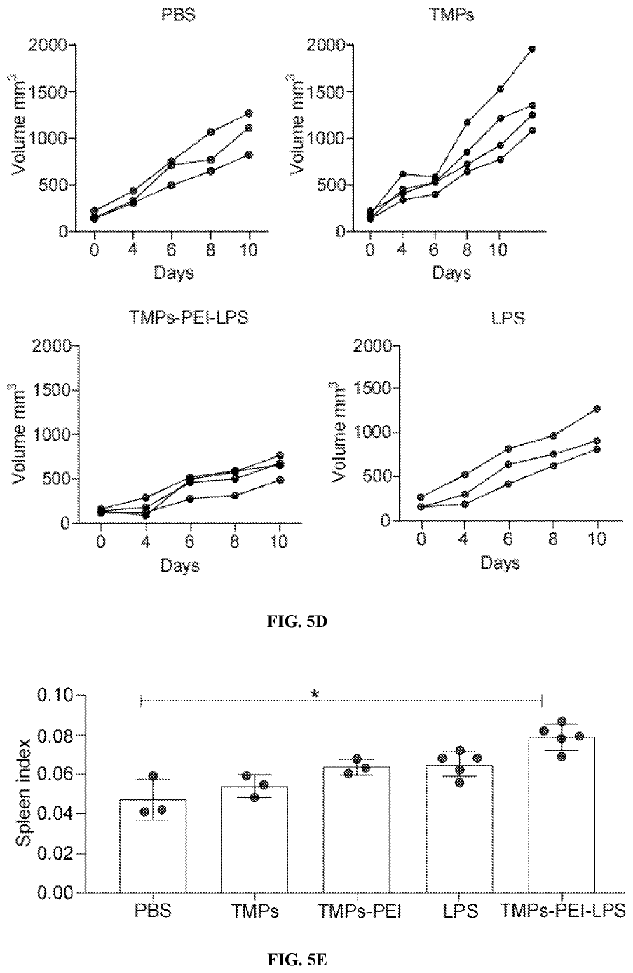

The anti-tumor effect of the TMP-PEI-LPS in vivo was verified by a mouse subcutaneous tumor model (FIG. 5A). Tumor-bearing mice were injected with PBS, MPs, LPS, and TMP-PEI-LPS by tail vein separately. At the end of the experiment, it was found that the tumor size and tumor growth rate of the TMP-PEI-LPS group were lower than those of the other groups, while body weight was not affected. This suggested that the TMP-PEI-LPS had a desirable effect on promoting tumor while showing safety. (FIG. 5A to FIG. 5C). The spleen index of the mice in each group was evaluated, and it was found that the spleen index of the TMP-PEI-LPS group was significantly increased, suggesting that the TMP-PEI-LPS inhibited tumor growth by promoting the occurrence of anti-tumor immunity.

The above description is an example of the best implementation of the present disclosure, and all the parts which are not described in detail are the common knowledge of those of ordinary skill in the art. The protection scope of the present disclosure shall be subject to the content of the claims, and any equivalent transformation based on the technical enlightenment of the present disclosure shall fall within the protection scope of the present disclosure.

1. A preparation method of a tumor vaccine based on tumor-derived microparticles (TMPs), comprising the following steps:
step 1, subjecting tumor cells to separation to obtain TMPs;
step 2, mixing the TMPs with 0.02 μg/μl to 0.04 μg/μl of a polyethyleneimine (PEI) solution to allow a reaction for 10 min to 20 min, and washing the microparticles to obtain PEI-modified TMPs (TMP-PEI); and
step 3, mixing the TMP-PEI with 0.05 μg/μl to 0.15 μg/μl of a lipopolysaccharide (LPS) solution to allow cross-linking for 15 min to 1 h to obtain the tumor vaccine based on TMPs (TMP-PEI-LPS).

2. The preparation method of a tumor vaccine based on TMPs according to claim 1, wherein the TMPs are obtained by subjecting the tumor cells to ultraviolet irradiation and to separation by gradient centrifugation in step 1.

3. The preparation method of a tumor vaccine based on TMPs according to claim 1, wherein the reaction is conducted at 4° C. for 15 min, and the PEI solution has a concentration of 0.03 μg/μl in step 2.

4. The preparation method of a tumor vaccine based on TMPs according to claim 1, wherein the reaction is conducted at 4° C. for 30 min in step 3.

5. The preparation method of a tumor vaccine based on TMPs according to claim 1, wherein the LPS solution has a concentration of 0.1 μg/μl in step 3.

6. (canceled)

7. A TMP-based tumor vaccine prepared by the preparation method according to claim 1.

8. The TMP-based tumor vaccine according to claim 7, wherein the TMPs are obtained by subjecting the tumor cells to ultraviolet irradiation and to separation by gradient centrifugation in step 1.

9. The TMP-based tumor vaccine according to claim 7, wherein the reaction is conducted at 4° C. for 15 min, and the PEI solution has a concentration of 0.03 μg/μl in step 2.

10. The TMP-based tumor vaccine according to claim 7, wherein the reaction is conducted at 4° C. for 30 min in step 3.
